(19) United States
(12) Patent Application Publication
(10) Pub. No.: US 2025/0087343 A1
(43) Pub. Date: Mar. 13, 2025

(54) ARTIFICIAL INTELLIGENCE APPOINTMENT SCHEDULING SYSTEM

(71) Applicant: Lightning Comm LLC, Montebello, NY (US)

(72) Inventors: Joshua L. Fox, Lawrence, NY (US); Jacob Baldinger, Monsey, NY (US)

(21) Appl. No.: 18/882,727

(22) Filed: Sep. 11, 2024

Related U.S. Application Data
(63) Continuation of application No. 18/641,464, filed on Apr. 22, 2024.
(60) Provisional application No. 63/537,554, filed on Sep. 11, 2023.

(51) Int. Cl. G16H 40/20; G06N 20/00; G06Q 10/02; G10L 15/22

(52) U.S. Cl. CPC G16H 40/20; G06N 20/00; G06Q 10/02; G10L 15/22

(57) ABSTRACT

A solution for patient scheduling systems, designed to streamline the appointment booking process and enhance the overall patient experience is provided. The solution may include a kiosk computer system that interacts with a patient using voice recognition technology. The solution may include an appointment booking system. The appointment booking system may include an interactive voice response system, a middleware layer and an electronic health record system. The system may also include an artificial intelligence model that is less susceptible to hallucinations. provide a seamless and efficient appointment booking process. The solution may adapt to office-specific requirements and improve patient satisfaction and practice efficiency.

### CROSS-REFERENCE TO RELATED APPLICATIONS

This application claims priority to U.S. Patent Application No. 63/537, 554, filed on Sep. 11, 2023, which is hereby incorporated by reference herein in its entirety. This application is also a continuation of U.S. patent application Ser. No. 18/641, 464, filed on Apr. 22, 2024, which is hereby incorporated by reference herein in its entirety.

### FIELD OF TECHNOLOGY

The present disclosure relates to a system for improving workflow in a healthcare facility. The system leverages improved artificial intelligence (“AI”) and multiple communication channels to provide patients with a dynamic, intuitive and efficient intake experience.

### BACKGROUND

Medical offices are notoriously hard to reach. Unless you have an urgent health problem that demands immediate attention, you must schedule an appointment to be seen by a doctor or obtain other medically related services. Scheduling an appointment can be very frustrating. According to the National Center for Health Statistics, in the United States there were over 1 billion physician office visits in 2019. See, https://www.cdc.gov/nchs/data/ahcd/namcs_summary/2019-namcs-web-tables-508.pdf.

When attempting to schedule an appointment, many patients report “operational friction” which includes long hold times, difficulty getting a timely appointment and trouble accessing follow-up information. Challenges scheduling a medical appointment are further because, despite a exacerbated proliferation of online scheduling tools, more than half of all medical appointments are still scheduled over the phone. In 2022, telephone calls were still the most preferred communication channel when a consumer needs help for any industry, even showing an 8% increase over 2021.

Healthcare patients are even more likely to pick up the phone. The majority of patients start their journey by searching for and researching providers online. However, after completing their research, patients typically attempt to schedule an appointment by calling. Although phone calls are highly preferred by patients, they are also one of the most frustrating communication channels. The front desk staff at a medical office are often juggling multiple tasks. They need to help book or change appointments, authenticate scheduled patients, verify insurance information, physically guide patients into the exam room. The staff also needs to field general questions regarding billing, payments, pharmacy prescriptions and other questions. A patient calling to schedule an appointment may need to speak with multiple staff members and suffer interminable hold time.

Conventional interactive voice response (“IVR”) systems do not provide any relief to this operational friction. IVR system may be powered by a pre-recorded messaging or text-to-speech technology utilizing a dual-tone multi-frequency (“DTMF”) interface. IVR systems may enable callers to receive or provide information, or make requests using voice or keypad inputs, without speaking to a live human agent.

A conventional IVR system is programmed to handle a predefined set of tasks and often does not provide flexibility for handling any other tasks. Consumers often feel frustrated when interacting with conventional IVR systems. Within conventional IVR systems, it is difficult to directly access a live human agent. Consumers cannot proceed directly to the task they need and typically waste time listening to irrelevant menu options. Finally, conventional IVR systems have difficulty understanding voice inputs received from consumers. Typically, consumers cannot interact with the conventional IVR system using complete sentences expressed in natural language, as consumers would do when speaking to a live human agent.

Recent advances in artificial intelligence (“AI”) have increased optimism that IVR systems may be able to provide a better consumer and patient specific experience. For example, conversational AI leverages large volumes of data, machine learning and natural language processing to help imitate human interactions, better recognizing speech and text inputs and translating their meanings across various languages. However, conversational AI systems also have several technological deficiencies.

Conversational AI systems are designed to collect information required to complete a task. The AI systems may utilize a large language model (“LLM”) to process information. However, this information still comes from contextual information or asking the caller a question. The caller may need to provide an 11-digit identification number, an address or name to accurately identify the caller. In voice environments, it may be challenging to correctly and accurately capture any of these data elements, even if the caller knows them and despite utilizing an LLM. For example, the spelling of given or surnames is a very challenging task, even for sophisticated LLMs.

AI systems are also vulnerable to hallucinations. A hallucination occurs when an AI model perceives patterns or inputs that are nonexistent or imperceptible to humans, creating outputs that are nonsensical or inaccurate. Preventing hallucinations is difficult. Some notable examples of AI hallucination include:
Incorrect predictions, such as predicting that an event will occur when it is unlikely to happen. An AI model used to predict the weather may predict that it will rain tomorrow when there is no rain in the forecast.
False positives, such as identifying something as being a threat or a fact when it is not. An AI model may concoct nonexistent company return, refund or cancellation policies.
False negatives, such as failing to identify something as being a threat when it is. An AI model used to detect cancer may fail to identify a cancerous tumor.

Hallucinations, which at times may be comical, are serious concerns for mission-critical applications such healthcare. Healthcare applications require reliable inputs and consistent outputs. This need has created a technological challenge. On the one hand, patients want and need automated solutions to improve access to and delivery of healthcare services. On the other hand, conventional automated solutions are unable to reliably interact with patients in a natural and conversational way that meets the standard high needed for healthcare applications.

Accordingly, it would be desirable to provide an improved AI solution that is less prone to hallucinations and allows patients to reliably interact with automated systems using conversational, natural language. Accordingly, it is desirable to provide apparatus and methods for an ARTIFICIAL INTELLIGENCE APPOINTMENT SCHEDULING SYSTEM.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows an illustrative system architecture in accordance with the principles of the description;

FIG. 2 shows an illustrative process in accordance with the principles of the description;

FIG. 3 shows an illustrative process and system components in accordance with the principles of the description;

FIG. 4 shows an illustrative process and system components in accordance with the principles of the description;

FIG. 5 shows an illustrative process and system components in accordance with the principles of the description;

FIG. 6 shows an illustrative system architecture in accordance with the principles of the description;

FIG. 7 shows an illustrative graphical user interface in accordance with the principles of the description;

FIG. 8 shows an illustrative graphical user interface in accordance with the principles of the description;

FIG. 9 shows an illustrative graphical user interface in accordance with the principles of the description; and

FIG. 10 shows an illustrative graphical user interface in accordance with the principles of the description.

### DETAILED DISCLOSURE

AI powered IVR technology has numerous practical healthcare applications. AI powered IVR systems may efficiently implement pre-treatment questionnaires, patient satisfaction surveys, lab and appointment scheduling, appointment follow-up and patient monitoring. However, in the context of healthcare applications such AI powered solutions must be more reliable and less susceptible to hallucinations.

Hallucinations are more likely when callers are able to freely provide input in a natural language conversational way. Hallucination risk may be reduced if the options for interacting with the AI are limited or scripted. However, such interactive limits may cause automated systems to be less conversational and function similar to conventional IVR systems. Apparatus and methods are provided for a system that reduces hallucinations in software that incorporates an AI model. Apparatus may include one or more features of a computer system.

Only three out of 10 consumers s who try to book a healthcare appointment online will succeed. It has been reported that more than half of patients are going without care access because the online appointment scheduling systems they rely on are not fulfilling their needs and expectations. Particularly, many patients report that they did not access healthcare in the past year because using an online self-scheduling system was too complicated. In one survey, 70 percent of patients reported that they began booking an appointment using an online self-scheduling tool or patient portal and were eventually referred to a telephone call with a call center staffer to finish booking. However, traditionally, booking an appointment over the telephone has been a long-winded and inefficient process.

On the other hand, humans naturally communicate using voice and it is an intuitive and fast method of communication. Apparatus described herein provide an automated voice booking system that is faster and more intuitive than online booking platforms. The voice-based system described herein eliminates waiting to connect with someone on the phone, getting placed on hold, or experiencing mistakes about available appointments or which insurance is covered.

The voice-based appointment booking system described herein is HIPPA compliant. The voice-based appointment booking system operates (e.g., respond to caller input, access and present information extracted from an EHR system) within time and performance limits that allow appointments to be booked in under five minutes and preferably under 3 minutes. The voice-based appointment booking system may mimic live human-to-human interaction and provide an institute comfortable interface for callers.

Apparatus may include an AI interactive voice recognition (“AI²VR”) system. The AI²VR system may include one or more features of a computer system. The AI²VR system may be configured to receive voice input from a caller. The AI²VR system may allow the caller to provide voice inputs even while a voice prompt is being presented to the caller. This may be referred to as “barging.”

The voice input received from a caller may be a natural language statement of the caller. The AI²VR system may be configured to compute an intent of the caller for each voice input reviewed from the caller. A caller's “intent” may refer to a recognition or understanding of what a caller wants to accomplish with a provided voice input. Understanding a caller's intent allows the AI²VR system to provide personalized and meaningful responses. By providing accurate and reliable intent recognition, unlike a conventional IVR system, an AI²VR system does not need to offer callers a fixed menu of options. Instead, the AI²VR system can ask open-ended, natural language questions such as, “How can I help you today?”

Computing an intent may include determining how a caller's voice input relates to content of a prompt presented by the AI²VR system. If the voice input includes a number, the AI²VR system may determine whether that number is part of an address, a date or an amount. The AI²VR system may determine whether the caller intended to say “to,” “too,” or “two.” The AI²VR system may also determine, based on the context associated the presented prompt, that despite recognizing an input as “too,” the caller likely meant to say “two.”

Computing an intent may include determining contextual awareness for a caller's voice input. Contextual awareness may be determined based on prior interactions between the AI²VR system and a caller. For example, the AI²VR system may determine that callers having a target demographic profile typically desire appointments having a predefined template. The predefined template may include one or more appointment selection parameters. Illustrative parameters may include a target calendar date, day of week, time of day, provider or location. When a caller that fits the demographic profile accesses the AI²VR system, the AI²VR system may automatically greet the caller and offer a set of available appointment slots that fit the predefined template.

The caller's intent may include a description of when a caller wants an appointment. For example, the AI²VR system may receive a natural language input from the caller that includes a request for an appointment and one or more parameters describing a desired appointment. For example, the one or more parameters may include a calendar date, day of week, time of day, provider or location. The AI²VR system may parse the parameters from the natural language description. The AI²VR system may formulate a search for available appointment slots that match one or more of the extracted parameters. The AI²VR system may apply one or more business rules that assign specific weights to the extracted parameters. The AI²VR system may present the available appointment slots to the caller for selection. The AI²VR system may schedule an appointment for the caller based on the determined selection.

Apparatus may include an AI model. The AI model may include one or more features of a computer system. The AI²VR system may include the AI model. The AI model may utilize predictive analytics to compute contextual awareness. By analyzing past call data, an AI model may detect patterns, such as peak call times or common caller queries. The detection of these patterns may allow the AI model to make predictions and proactive adjustments to the AI²VR system. The predictions and adjustments may allow the AI²VR system to provide faster response times and reduced wait times for callers. For example, the AI model may predict patterns, such as peak call times or common customer queries and dynamically activate an AI²VR system to assist answering calls in anticipation of those peak call times.

Computing an intent may include activating a service that fulfills the caller's needs. Activating the service may include linking disparate data sources to respond to a caller's request. Illustrative disparate data sources may include financial, health, employee, product, enterprise, inventory, weather, or any number of internal and external data sources.

The AI model may be provided by a commercial vendor, such as AI models and services provided by OpenAI, L.L.C. located at 3180 18th St. San Francisco, CA 94110 (e.g., ChatGPT or InstructGPT) or those provided by Google LLC located at 1600 Amphitheatre Parkway Mountain View, California 94043 USA (e.g., BARD).

An AI model may utilize one or more machine learning algorithms. An illustrative machine learning algorithm may include linear regression. Linear regression is a statistical method that models a relationship between a dependent variable and one or more independent variables. The linear regression algorithm is configured to compute a straight line that best fits between multiple data points. Linear regression is widely used for forecasting, trend analysis, and risk assessment in various fields.

An illustrative machine learning algorithm may include logistic regression. Similar to linear regression, logistic regression is typically used to model a relationship between a dependent variable and one or more independent variables. However, the dependent variable in logistic regression is categorical, meaning it can only take on a limited number of values (e.g., 0 or 1, yes or no). This makes it useful for classification tasks, such as spam filtering or predicting customer churn.

An illustrative machine learning algorithm may utilize one or more decision tree algorithms. A decision trees algorithm uses a tree-like structure to classify data. The inputs to the algorithm include responses to a series of questions, and based on the responses, determines a branch in the tree that leads to a final classification of the response. Decision tree algorithms are popular for their interpretability, because it is generally relatively easy to understand the logic behind their decisions.

An illustrative machine learning algorithm may include a naive bayes algorithm. A naive bayes algorithm is part of a family of generative machine learning algorithms and is based on the Bayes theorem, which calculates the probability of an event happening given some evidence. The naive bayes algorithm is computationally efficient and works well for many classification tasks, especially when dealing with large datasets.

An illustrative machine learning algorithm may include a K-Nearest Neighbors (“KNN”) algorithm. The KNN algorithm is optimized for classification and regression tasks. It operates by classifying a new data point based on the labels of its nearest neighbors in the training data. The number of neighbors (e.g., “K”) is a parameter that can be tuned to improve the performance of the model.

An illustrative machine learning algorithm may utilize one or more support vector machines (“SVM”). SVMs are powerful classification algorithms that can be used for a variety of tasks, including image recognition, natural language processing, and bioinformatics. SVMs operate by finding a geometric hyperplane that best separates the data points of different classes. A hyperplane is a generalization of a two-dimensional plane in three-dimensional space to mathematical spaces of arbitrary dimension. SVMs aim to find the best hyperplane that maximizes the margin between support vectors, enabling effective classification even in complex, non-linear scenarios.

An illustrative machine learning algorithm may include deep neural networks (“DNNs”). DNNs are modeled based on the structure of the human brain and are capable of learning complex patterns from data. DNNs learn by progressively transforming the input data through multiple layers of interconnected neurons. The weights assigned to these connections are tuned during training to achieve the desired output. DNNs are typically used for tasks such as image recognition, speech recognition, and machine translation.

An illustrative machine learning algorithm may include a random forests algorithm. A random forest algorithm is an ensemble learning technique that combines multiple decision trees to provide robust and accurate responses. The random forest algorithm takes a random sample of features at each split in the decision tree, which helps to reduce overfitting. Random forest algorithms are popular for classification tasks, especially when dealing with high-dimensional data.

An illustrative machine learning algorithm may utilize gradient boosting techniques. Like random forest, gradient boosting is also an ensemble learning method. Gradient boosting builds an AI model in a stage-wise fashion, where each stage aims to improve upon the predictions of the previous stage. The AI model building process starts with a base model, often a decision tree. Gradient boosting builds upon insights from each previous model's output to progressively refine the overall ensemble's predictions. Gradient boosting is a powerful technique that can be used for both classification and regression tasks.

An illustrative machine learning algorithm may include transformers. Transformers are a deep learning architecture often used for natural language processing (“NLP”). Transformers are based on the concept of self-attention, which allows the model to attend to different parts of the input sequence and learn long-range dependencies. Transformers have achieved remarkable results on a variety of NLP tasks, such as machine translation, text summarization, and question answering.

The AI model may be configured to receive a caller's voice input. The AI model may be configured to compute two or more intents of the caller based on the voice input received from the caller. The AI model may use one or more machine learning algorithms to derive an intent of the caller based on the received voice input.

Apparatus may include a middleware layer. The middleware layer may include one or more features of a computer system. The middleware layer may be configured to formulate a response to the caller based on one more intents computed by the AI model. The middleware layer may be configured to arbitrate between a first and a second intent. The middleware layer may be configured to limit risk of hallucinations by comparing a second intent to a first intent. The second intent may be computed by the AI model. The first intent may be selected from a defined set of expected responses.

The computational options available within the set of expected responses may be more tightly controlled than the computational options available to the AI model. By design, a human programmer may limit the set of expected responses. For example, the defined set of responses may be selected based on looking for target keywords within the caller's voice input. Using the set of expected responses as a control on the output of the AI model may provide a more reliable system that is suitable for use in mission-critical healthcare applications.

The middleware layer may be configured to throttle a level of regularization applied to the AI model. The middleware layer may determine a level of regularization or a specific regularization technique to apply to the AI model. The middleware layer may make a regularization decision based on a confidence measure associated with an intent of the caller.

The middleware layer may compute a confidence measure for an intent generated by the AI²VR system. The computed confidence measure may include a confidence level. The confidence level may represent a probability that an intent is an accurate interpretation of the voice input provided by the caller. The confidence level is typically expressed as a percentage. For instance, a 95% confidence level indicates that if the same voice input was received multiple times, the AI²VR system would generate the “true” intent 95% of the time. For a voice input, a “true” intent may refer to how a live human agent would have understood the caller's voice input.

The computed confidence measure may include a confidence interval. The confidence interval refers to generated intent that likely contains the “true” intent of the caller's voice input. For example, a 95% confidence interval means the AI²VR system has a 95% chance of generating an intent that includes the caller's “true” intent.

Regularization refers to a set of techniques used by machine learning algorithms to prevent an overfitting problem. Typically, an AI model relies on one or more machine learning algorithms to make a prediction. The AI model m provided a training dataset and programmed to learn and detect underlying patterns in provided data and use those patterns to make accurate predictions on new, unseen datasets. Overfitting occurs when an AI model memorizes the training data too well. The AI model behaves like a student who can only recite memorized answers but can't solve new problems that require applying concepts. Overfitting leads to poor performance on unseen data.

Regularization techniques act like “training wheels” for the AI model. Regularization adds constraints or penalties that prevent the AI model from becoming overly complex and fixated on every tiny detail in the training dataset. Regularization encourages the AI model to focus on capturing the general patterns that are more likely to generalize well to new datasets. Regularization may simplify the model by penalizing AI models that develop too many parameters or features. This discourages the AI model from becoming overly complex and fitting to random noise in datasets. Regularization may add a penalty term to the AI model. The AI model may then try to minimize the penalty term during training, which pushes the AI model towards simpler solutions that achieve good accuracy without overfitting. Regularization techniques may include data augmentation. Data augmentation techniques may include artificially creating new variations in the existing training datasets. Exposing the AI model to a wider range of examples, configure the AI model to learn more generalizable patterns.

Regularized AI models perform better on unseen data because they haven't memorized the training data to a fault. Regularization also helps to stabilize an AI model's performance, making it less sensitive to small changes in the training data. Regularization helps the AI model avoid providing responses that are very specific to the training data.

The middleware layer may be configured to pass the response it formulates to the AI²VR system. The AI²VR system may be configured to generate audio output based on the response. The AI²VR system may utilize conversational AI to generate the response provided to the caller. Conversational AI may refer to a computer program that provides a conversational experience that mimics actual conversations with another human.

In a conversational AI system, the AI²VR system may use an AI model to understand the customer's intent and respond with relevant information. Conversational AI allows the AI²VR system to provide responses to caller questions quickly and efficiently without requiring the callers to navigate through multiple menu options as in a conventional IVR system. Conversational AI allows the AI²VR system to provide callers open-ended prompts and compute accurate intents of caller responses to those open-ended prompts.

The conversation AI system may allow callers to communicate with an AI²VR system naturally and using complete sentences. In a conversational AI system, the AI²VR system may respond to a caller using natural language speech. The natural language speech output provided by the AI²VR system may be pre-recorded or generated dynamically.

The conversation AI system may include speech-to-text (“STT”) and text-to-speech (“TTS”) technology. The AI²VR system may capture a voice input provided by a caller and use STT technology to convert the captured voice input into text. After the middleware layer generates a response to the voice input, TTS technology may be used to convert a text-based response into speech output. SST and TTS technology may account for different languages, dialects, and regional accents. An AI-model coupled to STT and TTS technology allows an AI²VR system to engage in human-like conversations and generate lifelike speech that express emotion and vocal nuance.

The middleware layer may be configured to increase the level of regularization based on a threshold confidence measure associated with the first intent. The middleware layer may decrease the level of regularization when the first intent is above a threshold confidence measure.

The middleware layer may be configured to instruct the AI model to compute the second intent before the AI²VR system computes the first intent. The middleware layer may compute a confidence measure associated with the second intent. When the confidence measure associated with the second intent is below a threshold level, the middleware layer may instruct the AI²VR system to compute the first intent.

The AI²VR system may be configured to compute an intent based on a fixed set of responses that have been reviewed by a human programmer. The human programmer may use the AI model to generate the fixed set of responses. However, the fixed set of responses may only be added to the AI²VR system after review by the human programmer. The intent generated by the AI²VR system may be immune from hallucinations. Computing two intents for a voice input by two different systems (AI²VR system and AI model) may reduce the risk of acting on any hallucinations generated by the AI model.

The middleware layer may be configured to instruct the AI²VR system to compute the first intent. In some embodiments, the middleware layer may assess a confidence measure associated with the first intent. When the confidence measure associated with the first intent is below a threshold level, the middleware layer may instruct the AI model to compute the second intent. By first computing the second intent, the middleware layer may compare the AI generated second intent to a first intent computed by the AI²VR system based on a fixed set of responses that have been reviewed by a human programmer.

The AI model may be configured to recursively train itself using a plurality of first intents generated by the AI²VR system. For example, the AI model that includes gradient boosting may utilize the plurality of first intents to train a new decision tree. The first intents computed by the AI²VR system may be based on a fixed set of responses that have been reviewed by a human programmer. Using the first intents as training data may further prevent the AI model from generating a hallucinatory second intent.

In some embodiments, the AI model may be configured to generate a plurality of intents in response to a prompt presented to a caller. The AI²VR system may be configured to determine whether a voice input received from a caller matches one of the plurality of intents generated by the AI model. Thus, rather than the AI model alone to decide an intent of the caller, the AI²VR system may decide whether the intent generated by the AI engine is non-hallucinatory. Such oversight by the AI²VR system may further prevent the AI model from generating a hallucinatory intent or associated response.

The AI model may be configured to activate the AI²VR system and the middleware layer in response to detecting a pre-defined trigger event. The pre-defined trigger event may be an inclement weather event at a target location. For example, if the AI model detects a snowstorm at a target location, the AI model may activate the AI²VR system to answer calls dialing the target location because it is likely that the target location will be understaffed due to the inclement weather.

The trigger event may be a time outside regular business hours such as nights, holidays, or weekends. The AI model may activate the AI²VR system to answer calls dialed during times when employees are typically not available to answer calls.

Apparatus may include a computer program. The computer program may include instructions that when executed on a processor on a computer system, cause the computer system to perform one or more functions or actions.

The computer system may include hardware and software. Hardware may include a Central Processing Unit (“CPU”). The CPU may be responsible for processing instructions and performing calculations. The CPU may control overall speed and performance of the computer system. The hardware may include Random Access Memory (“RAM”). RAM typically refers to temporary storage that holds data that the CPU is actively using. RAM allows the CPU to quickly access frequently used information.

Hardware may include a storage device which data persistently even when the computer system is powered off. Illustrative storage devices may include hard disk drives (HDDs) and solid-state drives (“SSDs”). SSDs typically offer faster data access compared to HDDs. Hardware may include a motherboard. The motherboard is a central circuit board that connects all the other hardware components and allows them to communicate with each other. For example, the motherboard may connect the CPU to a Graphics Processing Unit (“GPU”). GPUs, while traditionally used for video processing, are great at handling complex mathematical tasks. GPUS are increasingly important for accelerating demanding applications like video editing and machine learning algorithms.

Hardware may include input and output devices. Input devices allow human users to interact with the computer system. Illustrative input devices include keyboards, mice, touchscreens, webcams, and microphones. Output devices display information processed by the computer. Illustrative output devices include monitors, printers, and speakers.

The computer system may include software. Software may include a set of instructions that tell the hardware what to do and how to function. Software may include system software and application software.

System software manages a computer system's resources and provides a platform for running other programs. Examples include operating systems (like Windows, macOS, or Linux) and device drivers that control the operation of specific hardware components part of the computer system. Application software allows users of the computer system to perform specific tasks. Examples of application software include web browsers for browsing the internet, word processors for creating documents, and video games for entertainment.

A computer program may include instructions that configure a computer system to answer a telephone call from a caller. The instructions may prompt the caller to request a service that requires integration with an electronic health record (“EHR”) system. The instructions may communicate with the EHR system and provide the requested service to the caller during the telephone call.

An illustrative EHR system may electronically store medical information about a patient in a secure and centralized location. The EHR system may electronically store patient information such as demographics, medical history, allergies, medications, and immunizations. The EHR system may electronically store clinical data such as doctor's notes, test results, radiology images, diagnoses, and treatment plans. The EHR system may include tools to support care such as drug interaction checking, clinical decision support systems, and patient portals.

The instructions may prompt the caller using natural language and lifelike speech. Lifelike speech may include computer generated speech that expresses emotion and vocal nuance. Lifelike speech may mimic speech in a human-to-human conversation.

The computer program may generate lifelike speech using an AI model that converts text into lifelike speech. The computer program may prompt the caller using the lifelike speech. The computer program may convert the text into lifelike speech by generating, based on a computed meaning of the text, natural sounding: intonation, resonance, voice and tone.

The computer program may prompt the caller in a first language. In response to detecting a voice input provided by the caller in a second language, the computer program may interact with the caller in a second language.

The computer program may receive a voice input from the caller and determine whether the voice input includes a unique speech signature associated with the caller. In response to detecting the unique speech signature in the caller's voice input, the computer program may locate a record corresponding to the caller and stored in the EHR system.

Conventional IVR systems and call centers use a time-consuming knowledge-based authentication process. Callers are asked to their social security number, date of birth, or mother's family name. The computer program may utilize an AI model to provide real-time voice-based authentication. The computer program may analyze a caller's voice characteristics and provide a real-time authentication decision of the caller's identity.

The computer program may analyze the caller's speech characteristics such as rhythm, pitch, and tone to create a unique speech signature for a caller. When a voice input is subsequently captured from the caller, the computer program may automatically attempt to match the voice input to the stored unique speech signature. If the voice input matches the unique speech signature, the caller is authenticated. The caller does not say any specific phrases or words or otherwise interrupt a natural conversation flow.

The computer program may receive a service request from a caller. In response to receiving the service request, the computer program may automatically formulate an Application Programming Interface (“API”) call. The API call may be formatted in accordance with one or more requirements of an EHR system. The computer program may receive a response to the API call from the EHR system. The computer program may extract data from the response provided by the EHR system and present the extracted data to the caller. The computer program may reformat the data for consumption by the caller.

The computer program may present the extracted data to the caller during a telephone call using lifelike speech. The computer program may present the extracted data to the caller during a telephone call via text message. The computer program may present the extracted data to the caller during a telephone call using a first communication channel and a second communication channel. The first communication channel may present the extracted data to the caller as speech. The second communication channel may present the extracted data to the caller as a text message.

The computer program may facilitate bi-directional communication between an AI²VR system that answers the telephone call and the EHR system. The computer program may facilitate bi-directional communication between the AI²VR system and two or more EHR systems. The computer program may facilitate bi-directional communication between two or more AI²VR systems and the EHR system.

During a telephone call, the computer program may capture at least two data elements that uniquely identify the caller. The caller may provide the two data elements as one or more voice inputs. The caller may provide the data elements as natural language conversational speech. An illustrative data element may include a birthdate. An illustrative data element may include a telephone number. An illustrative data element may include a caller's first name. An illustrative data element may include a caller's last name.

Based on at least two data elements, the computer program may formulate an API call to search in the EHR system for a patient record corresponding to the caller. In response to locating the patient record, the computer program may provide a requested service to the caller using information stored in the patient record. In response to failing to locate the patient record, the computer program may formulate a second API call. The second API call may create, within the EHR system, a new patient record corresponding to the caller. The newly created patient record may include the two data elements captured from the caller.

The computer program may concurrently answer multiple telephone calls from multiple callers. The computer program may answer each telephone call after fewer than three rings. The computer program may be configured to limit a duration of the telephone call to 5 minutes or less. The computer program may be configured to provide the requested service to the caller in a phone that lasts 5 minutes or less.

Illustrative services that may be requested by a caller and provided by the computer system may include booking a new appointment, cancelling an appointment or rescheduling an appointment. The computer program may customize the service for the caller. For example, based on contextual awareness the computer program may search for available appointment slots that conform to a target appointment template. The target appointment template may include a target date, time, provider or location. Based on the patient record extracted from the EHR system, the computer program may automatically greet the caller by name and offer the earliest available appointment that fits the target appointment template.

The computer program may customize the service for the caller by formulating an API call to the EHR system based on inputs captured from the caller during the telephone call, information extracted from the EHR system and optimization criteria set by a medical office that uses the EHR system.

Illustrative services that may be requested by a caller and provided by the computer system may include responses to requests for general office information. Such requests may include whether there is parking available, office hours, insurance plans accepted and providers that practice at a location. The computer system may provide appropriate responses to caller inquiries. Such responses may be generated in natural language by an AI engine.

Apparatus may include an automated system for scheduling an appointment during a phone call. The automated system may include an AI²VR system. The AI²VR system may be configured to answer a phone call received from a caller. The AI²VR system may be configured to concurrently answer multiple phone calls received concurrently from a plurality of callers. The AI²VR system may be configured to begin interacting with each caller within a threshold time limit. The threshold time limit may be defined based on a number of rings before the AI²VR system answers and begins interacting with a caller.

The AI²VR system may be configured to receive voice input from a caller. The voice input may be provided by the caller in natural language. The AI²VR system may be configured to accept voice input in any of 30+ languages. The AI²VR system may be configured to provide audio output in any of the 30+ languages. The AI²VR system may provide the audio output in lifelike speech.

The automated system may include a middleware layer. The middleware layer may be configured to schedule an appointment for the caller based on the voice input. The middleware layer may be configured to schedule the appointment via an integration with an electronic health record (“EHR”) system. The middleware layer may be configured to integrate with a plurality of EHR systems.

The AI²VR system may be configured to authenticate the caller using the voice input. The AI²VR system may be configured to analyze voice characteristics of a second voice input received from the caller. The voice characteristics may include rhythm, pitch, and tone, timbre, intensity, accent, pronunciation or cadence of voice input captured from the caller.

Based on the voice characteristics, the AI²VR system may generate a digital voiceprint corresponding to the caller. The AI²VR system may utilize the digital voiceprint to perform a real-time identify decision of the caller in response to receiving the first voice input.

The AI²VR system may be configured to extract information from the voice input received from the caller. The middleware layer may be configured to use the extracted information to formulate a search for a patient record within the EHR system. For example, the extracted information may include the caller's name, birthdate, insurance information, address, phone number or desired provider. The middleware layer may be configured to create a new patient record within the EHR system when a patient record is not located within the EHR system.

The voice input received from the caller may be a first voice input. When the patient record is not located within the EHR system, the middleware layer may trigger the AI²VR system to prompt the caller for a second or additional voice inputs. The middleware layer may formulate additional searches for the patient record within the EHR system using information extracted from the second or additional voice inputs.

Based on the voice input captured by the AI²VR system, the middleware layer may be configured to cancel an appointment for the caller in the EHR system. Based on the voice input captured by the AI²VR system, the middleware layer may be configured to reschedule an appointment for the caller in the EHR system.

The middleware layer may be configured to extract at least one available appointment slot from an EHR system. The AI²VR system may be configured to send a text message to the caller that includes details of the available appointment slot. The AI²VR system may be configured to present the caller with an audio description of the details of the available appointment slot. The caller may be presented with both the audio description and the text message that includes details of the available appointment slot.

The voice input received from the caller may include insurance information. The AI²VR system may extract insurance information from the voice input. AI²VR system may pass the extracted insurance information to the middleware layer. The middleware layer may be configured to search for an available appointment slot with a service provider that is associated with the insurance information. The middleware layer may be configured to only present the caller with available appointment slots for providers that accept the caller's insurance.

The AI²VR system may extract a date and time from the voice input received from the caller. AI²VR system may pass the extracted date and time to the middleware layer. The middleware layer may be configured to search for an available appointment slot for the caller on or within a threshold temporal distance of the received date and time. An illustrative threshold temporal distance may be within 36 hours or within 60 days of the date and time provided by the caller.

The middleware layer may be configured to search for an available appointment slot for the caller based on a previous appointment scheduled by the caller. For example, the middleware layer may locate a patient record and examine previous appointments scheduled by the caller. The middleware layer may formulate a template that can be used to search or filter for available appointment slots that match the template. An illustrative template may include a target provider and a target day of the week. An illustrative template may include any suitable number of parameters.

The middleware layer may be configured to search for an available appointment slot for the caller based on a demographic characteristic of the caller. The demographic characteristic may be extracted from a patient record retrieved from the EHR system. The middleware layer may be configured to search for an available appointment slot for the caller based on a plurality of appointments previously scheduled by other callers that each share one or more demographic characteristics in common with the current caller.

The middleware layer may be configured to search for an available appointment slot for the caller that has a target duration. The middleware layer may determine the target duration based on a demographic characteristic of the caller. The middleware layer may determine the target duration based on a type of service requested by the caller. The type of service may correspond to an appointment type. The type of service may be extracted by the AI²VR system from the voice input received from the caller. The type of service may be determined based on a reason for scheduling the appointment provided by the caller. The caller may provide the reason in natural language. The AI²VR system may correlate the reason provided by the caller in natural language to one or more predefined service or appointment types. Illustrative service or appointment types may include cosmetic, medical, follow up care, new patient or emergency service.

The middleware layer may be configured to allow overbooking when scheduling an appointment for the caller. The middleware layer may be configured to determine whether to allow overbooking based on a demographic characteristic of the caller, a provider associated with the appointment, and/or a date and time of the appointment. For example, some providers may be comfortable when patients are overbooked. Other providers may feel overwhelmed when patients are overbooked. Based on the provider assigned to the appointment, the middleware layer may determine whether to overbook an appointment slot.

The middleware layer may be configured to assign a weight to one or more available appointment slots. The assigned weight may be determined based on weights assigned to appointment scheduling parameters that define the appointment slot. The middleware layer may formulate a set of appointment slots for the caller based on the relative weight assigned to a plurality of available appointment slots. The middleware layer may be configured to assign a weight to an available appointment slot based on an attribute of a prior appointment scheduled by the caller. The middleware layer may pass the formulated set of available appointment slots to the AI²VR system. The AI²VR system may present the set of available appointment slots to the caller using one or more communication channels.

The middleware layer may formulate a set of available appointment slots based on a set of filtering rules. The filtering rules may be configured to achieve a target optimization in a schedule. The filtering rules may be configured to formulate at least one appointment slot is acceptable to the caller. An illustrative target optimization may include grouping scheduled appointments into predefined blocks of time. An illustrative target optimization may include presenting the caller with an appointment slot that is not likely to be missed or cancelled by the caller.

The filtering rules may be programmable from within a user interface associated with the middleware layer. Illustrative filtering rules may be set for different caller profiles, office locations, providers, times of day or combinations thereof. For example, the middleware layer may be configured to present available appointment slots to the caller based on being programmed to fill a target block of time for a target provider at a target office location.

The middleware layer may include an AI model that dynamically adjusts the filtering rules. The AI model may dynamically change the filtering rules for different callers or at different times of day. The filtering rules may be dynamically adjusted by the AI model based on real-time availability of appointment slots at an office location or with a specific provider. The filtering rules may be dynamically adjusted by the AI model based on a caller's voice characteristic. The voice characteristic may be determined based on real-time voice input received by the AI²VR system. The filtering rules may be dynamically set by the AI model based on a reason provided by the caller for requesting an appointment. The filtering rules may be dynamically set by the AI model based on a characteristic of a provider selected by the caller. Illustrative provider characteristics may include overbooking rate, average number of patients seen per day, average appointment length, specialty, next available appointment slot, insurance accepted, or any other suitable characteristic.

The AI²VR system may be configured to process voice input as expressed by a caller in natural language. The voice input provided by the caller and received by the AI²VR system may include recitation of a birthdate. The AI²VR system may be configured to extract a birthdate of the caller from the natural language voice input. An illustrative voice input may include one or more of a caller's: given name, last name, a birthdate, mailing address, desired provider, insurance carrier, desired appointment time (date, a day of the week and time of day).

The AI²VR system may be configured to convert the voice input received from a caller into text. The AI²VR system may utilize an AI model to convert the voice input into text. The AI²VR system may be configured to determine a spelling of the converted text using data obtained from a reverse phone lookup. A number used by the caller to dial the AI²VR system may be used as a search parameter for the reverse phone lookup. For example, a voice input provided by a caller may include a surname. The AI²VR system may be configured to correct or determine a spelling of the surname using the data obtained from the reverse phone lookup.

The AI²VR system may be configured to extract a desired appointment search parameters from voice input received from the caller. Illustrative extracted appointment search parameters may include provider, calendar date, time of day, day of week, appointment type and office location. The middleware layer may be configured to search for an available appointment slot that meets scheduling criteria expressed by the caller.

The AI²VR system may be configured to prompt the caller using an audio prompt expressed in natural language. An AI model may be configured to dynamically formulate content of the audio prompt. The AI model may generate content based on a context of the conversation. The AI model may adjust the content of a prompt presented by the AI²VR system based on the time of day or recent activity of the caller. The AI model may be configured to adjust a characteristic of a voice output presented by the AI²VR system based on any suitable characteristic of voice input received from the caller.

The middleware layer may be configured to activate a target communication channel. In some embodiments, the middleware layer may trigger the AI²VR to activate the target communication channel. In some embodiments, the AI²VR system may activate the target communication channel. The target communication channel may be used to transmit information to the caller during the phone call. An illustrative target communication channel may include one or more of a video call, a text message, an email message, an audio message, an automated bot and an online form.

The AI²VR system may be configured to initiate a primary communication channel during the phone call. The middleware layer may be configured to trigger activation of at least one secondary communication channel during the phone call. The primary communication channel and the secondary communication channel may be active concurrently. For example, the caller may be presented with details associated with available appointment slots in lifelike, natural language speech and in a text message.

The AI model may be configured to determine, during the phone call, whether to transfer the caller to a live human agent. The AI model may be configured to activate the target communication channel. The AI model may be configured to activate the target communication channel based on content that will be transmitted to the caller using the target communication channel.

For example, if the AI model determines that it is more intuitive for a caller to consume content in a text form, the AI model may send the caller a text message. If the AI model determines that it is more intuitive for the caller to consume the content verbally, the AI model may send the caller an audio message. The audio message may be generated and/or delivered by the AI²VR system. The AI model may be configured to dynamically control one or more characteristics of any voice output presented to a caller by the AI²VR system.

The AI model may be configured to dynamically adjust functionality of the middleware layer that is available to the caller during the phone call. The AI model may restrict whether the middleware layer allows the caller to utilize the AI²VR system for a target appointment related service. For example, if a caller has a history of no shows or frequent appointment cancellations, the AI model may not allow the caller to cancel an appointment using the AI²VR system.

The AI model may be configured to dynamically generate an appointment template for the caller. The appointment template may include a set of search parameters. Illustrative parameters may include appointment type, date, day of week, time and provider. The AI model may submit the appointment template to the middleware layer. The middleware layer may search for an available appointment slot for the caller that matches one or more of the search parameters included in the appointment template.

An AI model may utilize one or more of the following computational techniques: linear regression, logistic regression, decision trees, support vector machines, naive bayes, K-nearest neighbors, K-means, random forests, dimensionality reduction, gradient boosting and adaptive boosting. The AI models may utilize one or more of the following computational techniques: supervised machine-learning, unsupervised learning machine-learning, semi-supervised machine-learning and reinforcement machine-learning.

The AI model may be configured to search for appointment slots based on based on appointment history data associated with a caller. The appointment history may include a number of times the caller has missed previous appointments, a number of times the caller has rescheduled previous appointments, a number times the caller has cancelled the previous appointments, a number of times the caller has been late to the previous appointments, an amount of time the caller has been late to the previous appointments, a number of times the caller has been early to the previous appointments, an amount of time the caller has been early to the previous appointments, a number of times the caller has arrived on-time to the previous appointments, a total number of appointments scheduled on behalf of the caller, nature of services (e. g., elective or medically needed) associated with the appointment, monetary revenue expected to be generated by the appointment, caller behavior at the previous appointments and/or total duration of each of the previous appointments relative to scheduled time for each of the previous appointments and duration of appointments schedule for other patients. Each factor included in the appointment history may not be assigned an equal weight when the AI model determines appointment slots for a caller.

The middleware layer may generate a reminder for a patient about an upcoming appointment. The middleware layer may trigger the AI²VR system to transmit the reminder electronically to the patient (e.g., email or text message). The middleware layer may generate the reminder based on data of the patient stored in the EHR system and/or the practice management system. The middleware layer may generate the reminder based on appointment history data associated with the patient.

An illustrative appointment history data may include: a number of times the patient has not showed up to previous appointments, a number of times the patient has rescheduled previous appointments, a number times the patient has cancelled the previous appointments, a number of times the patient has been late to the previous appointments, an amount of time the patient has been late to the previous appointments, a number of times the patient has been early to the previous appointments, an amount of time the patient has been early to the previous appointments, a number of times the patient has arrived on-time to the previous appointments, a total number of appointments scheduled on behalf of the patient, a nature of services (e.g., elective or medically needed) associated with the appointment a monetary revenue expected to be generated by the appointment patient behavior at the previous appointments and/or total duration of each of the previous appointments relative to scheduled time for each of the previous appointments and duration of appointments schedule for other patients.

A method for electronically authenticating a caller that dials into an AI²VR system is provided. The method may include executing computer executable instructions on a processor that configures a computer system to perform one or more tasks. For example, the method may include biometrically authenticating a caller. The system leverage biometric authentication performed by a device of the caller. For example, a caller's smartphone may perform facial, iris or fingerprint identification. The AI²VR system may link the biometric authentication performed by the caller's device to a mobile number that was used to dial into the AI²VR system. The method may include prompting the caller to scan or capture a picture of their driver's license or other form of identification. The method may include extracting data from the driver's license or other forms of identification. Based on the data extracted from the driver's license or other form of identification, the methods may include confirming a name, an address and a date of birth of the caller.

Methods may include prompting the caller to scan an insurance card or other proof of insurance coverage. Methods may include extracting data from the insurance card or other proof of insurance coverage. Based on the data extracted from the insurance card or other proof of insurance coverage, methods may include determining insurance eligibility and insurance attributes for the caller. Based on the data extracted from the insurance card or other proof of insurance coverage, methods may include prompting the caller for a payment and presenting a target workflow to the caller.

Methods may include presenting the caller with information about procedures, products and/or services available in the healthcare facility. Methods may include presenting the caller with the information about procedures, products and/or services based on data stored in an EHR system and/or practice management system and associated with the caller or a demographic profile associated with the caller.

Methods may include presenting a caller with information about products, services, procedures, specials available in a facility. Illustrative facilities may include one or more of: a nursing home, a medical office, an outpatient surgery center, an urgent care center, a hospital, an assisted living facility, a skilled nursing facility and/or any other healthcare facility.

Methods may include generating the information presented to the consumer based on a need or goal of the facility. Methods may include generating the information presented to the caller based on a need or goal of the caller. Methods may include generating the information presented to the caller based on a need of the caller and the facility.

The AI²VR system may include support for conversing in multiple (e.g., 30+) languages such as English, Spanish, German, Chinese, Korean, French, Arabic and Hebrew. The AI²VR may use AI algorithms to interpret and compute intents for voice inputs provided by patients using natural, conversational language.

The AI²VR system may use AI algorithms to generate lifelike voices that mimic the voice, tone, expressions of a provider or other office staff member. The AI²VR system may attempt to derive a spoken pronunciation of a patient's name or any other word. The AI²VR system may record a desired pronunciation of a patient's name. The AI²VR system may transfer the desired pronunciation to the front desk or medical assistant, doctor or any office staff member.

The AI²VR system may “mimic” patient's desired pronunciation as accurately as possible (e.g., when greeting the patient in the exam room or at a subsequent visit). Mimicking the voice attributes of a provider may enhance credibility of the AI²VR system when communicating with a patient. The AI²VR system may use the voice of a famous cartoon character or other celebrity (e.g., President of the United States).

The middleware layer may query an EHR system. Based on responses from the EHR system, the middleware layer may pass information to the AI²VR system. The AI²VR system may utilize the information received from the middleware layer to express recognize, greet and interact with callers. The middleware layer may identify pre-existing patients. The middleware layer may identify new patients and register their biometrics so that they will be subsequently recognized by the middleware layer. The middleware layer may register new patients. The middleware layer may register a new patient by creating a new patient record within the EHR system.

The middleware layer may maintain a digital log of all activities conducted by the middleware or AI²VR systems. The log may track provided caller input. The log may track outputs provided by the middleware or AI²VR systems and internal operations of the middleware layer during an interaction. For example, the middleware layer may track and log the filters applied to generate a set of appointment slots. The middleware layer may track, and log appointment slots presented to a caller and which of the presented appointment slots were selected by the caller.

To protect patient personal health information (“PHI”) the middleware and AI²VR systems may be configured to perform a data wipe between calls or otherwise periodically remove PHI from temporary storage locations. The data wipe may remove all PHI stored in transient memory or non-transitory memory associated with the middleware and AI²VR systems. All data stored in one or more databases associated with the middleware and AI²VR systems may be encrypted to prevent unauthorized access. The middleware and AI²VR systems may employ security techniques such as BitLocker to prevent access to information even if there is an unauthorized removal of memory or hard drives.

The middleware or AI²VR systems may send messages (e.g., facsimile, text, WhatsApp, email, push notifications) to a caller's mobile device.

The middleware layer may track caller interest in presented products, procedures, services and promotions. The middleware layer may create a note or record so that a provider or other office staff can see what the caller was interested in and follow up with the middleware layer.

Based on information in the patient record or caller input, the middleware layer may trigger the AI²VR system to present videos, photos, before and after pictures and other information (e.g., pulled from medical journals or reliable internet sources) about a condition or complaint the caller is concerned about. The presented information may educate and calm the caller.

The AI²VR system may present information to a patient about a provider, such as credentials, experience, speaking engagements or authored journal articles. The AI²VR system may present information about appointment slots with other providers that are available at an office location.

The AI²VR system may accept payments from a caller. The AI²VR system may recognize a caller's voice and accept voice authorizations of payments using previously stored payment information. The AI²VR system may capture biometric information from the caller and thereby authenticate the AI²VR system and authorize payments.

The middleware layer may be configured to determine incentives (e.g., discounts, transportation or promotional products) to encourage a caller to schedule appointments during times/days that would otherwise be undesirable or are typically slow days at the office or other office/provider preferences. The middleware layer may use an AI model or other software to formulate incentives that are appropriate for a caller.

The middleware layer may be configured to generate appointment reminders. The middleware layer may account for a caller's past or current medical history. For example, a caller associated with a diagnosis (e.g., a life-threatening condition) or history of missing appointments may receive additional appointment reminders. When scheduling appointments or reminders the middleware layer may account for a caller's culture or socioeconomic factors.

The middleware layer may promote specials or other office products and procedures based on time of day/week/year and/or based on how much inventory is available or provider work schedules.

Deployment of the middleware layer may provide a medical office with functional advantages. The middleware layer may improve compliance with regulatory requirements and maximize reimbursement rates. The middleware layer may provide callers with better access to better information and curated appointment choices. The middleware layer may increase caller interaction with service, specials, and products available at an office or healthcare facility.

Apparatus may include an artificial intelligence interactive voice response (“AI²VR”) appointment booking system. The AI²VR system may include one or more features of a computer system. Appointment scheduling is a gatekeeping function that defines whether one can efficiently and timely access health services. Timely access to healthcare services is important for realizing good medical outcomes. The AI²VR system may provide efficient and natural interaction for booking appointments. The AI²VR system may facilitate access to healthcare for populations that would otherwise face challenges accessing such critical services.

The AI²VR system may be capable of conversing in multiple languages, allowing more callers to schedule appointments by interacting with a language they understand. The AI²VR system may apply business rules that more efficiently schedule appointments. The business rules may include a scheduling algorithm. The scheduling algorithm may be customized for an office or provider. Application of such scheduling algorithms may improve access to medical care.

For example, an illustrative business rule may include scheduling more appointments during certain times of day and less appointments during other times of day. For example, during morning hours, a provider may work faster and during early afternoon hours a provider may be sluggish. Comparatively more appointments may be scheduled during the morning hours and comparatively less appointments may be scheduled during the early afternoon hours. No overbookings may be allowed during the early afternoon hours.

For example, application of the business rules may provide more access to appointment slots that would have otherwise been unused. A medical office may often reserve appointments for specialty procedures. However, if those reserved appointments are not booked, they may remain empty and unused for any patient, representing a waste of available health care resources. Within a target time of a reserved appointment slot (e.g., 24-36 hours), the business rules may allow any medical service to be booked in a reserved appointment slot so that those slots do not remain unused.

Such business rules may be configured to allow target patients priority access to health care services. For example, business rules may allow overbooking in certain geographic areas or for patients that fit a target profile. The target profile may be defined by an AI model based on analysis of the patient's record stored in an EHR system. For example, the business rules may be configured to allow overbooking for a patient with an acute or life-threatening condition. The business rules may be configured to allow overbooking for a patient that lives in a rural area with limited access to healthcare services.

Application of smart scheduling rules may provide callers with appointments that they are less likely to cancel or miss. For example, the business rules may include using an AI model to derive a predefined appointment slot template for a caller. The template may include a target calendar date, day of week, time of day, provider or location. The AI model may generate the template based on previous appointments scheduled by the caller. The AI²VR system may automatically offer a set of available appointment slots that fit the predefined template. The template may increase the likelihood that the caller will be presented with an appointment slot that is a convenient and desirable choice for the caller. The caller may be less likely to cancel or change such an appointment. Reducing a no-show or cancellation rate may increase utilization of relatively limited healthcare resources.

The AI²VR system may include a middleware layer (Layer 2) that interacts rapidly with a telephony layer (Layer 1) that answers phone calls and assigns intents to caller voice inputs. The telephony layer may include one or more functions of a conventional IVR system. The middleware layer may integrate with one or more EHR systems (Layer 3). The AI²VR system may collectively include the telephony layer (Layer 1), middleware layer (Layer 2) and an integration to one or more EHR systems (Layer 3).

The AI²VR system may interact with callers using intuitive and natural language voice commands that substitute for caller interaction with a live, human agent. The AI²VR system may provide callers with voice prompts using natural, human sounding voices and in multiple languages. After initiating a voice-based conversation, the AI²VR system may facilitate bi-directional interaction with the caller.

The AI²VR system may allow the caller to shift back and forth between a text, video and/or voice conversation. The AI model may understand callers when they speak in full sentences using natural language. The AI model may decipher caller voice inputs. The AI model may machine-generate responses in lifelike speech to the caller voice inputs. The AI model may provide machine-generated responses that sound humanly conversational and include typical human fillers (such as “uhh” or “hmmm”), expression and intonation.

The AI model may preserve a conversation context. For example, while providing a voice input, a caller may pause mid-sentence to address something else. The AI model may detect the interruption and identify cues that the caller has reengaged with the conversation.

The middleware layer may apply slot filtering rules. The slot filtering rules may be customized by each office. The slot filtering rules may be customized for individual providers. For example, a provider may have scheduling preferences. Such preferences may include scheduling new patients in the afternoon and preexisting patients in the morning. Scheduling preferences may include filling a schedule starting at the last available appointment and working toward the first appointment of the day. Scheduling preferences may include scheduling target services on specific days of the week or month.

The slot filtering rules may impact various stages of the appointment booking process. The slot filtering rules may apply business rules that embody business intelligence logic for reducing no-shows and optimizing scheduling. The slot filtering rules may attempt to fill appointment slots on target days/times and provide callers with desirable appointment slots. The slot filtering rules may encourage patients to book appointments that are optimal for themselves, the office, and maximize utilization of healthcare resources.

The slot filtering rules may take account of a provider's scheduling preferences, time of day, and environmental factors such as weather and current traffic conditions. The slot filtering rules may dynamically adjust an overbooking rate based real-time or historical data. An illustrative slot filtering rule may generate a set of appointment slots for a caller based on: available appointment slots, office location(s), a provider's working schedule, the provider's scheduling preferences, whether a caller is a new or established patient, medical insurance coverage, desired provider (doctor, MA, PA, surgeon), desired date and time of appointment, and patient/caller demographic information. Other custom slot filtering rules may be configured by an office.

The AI²VR system (Layers 1, 2 and 3) will be HIPPA compliant. The AI²VR system may integrate with one or more EHR systems. Each EHR system integration may allow the middleware layer to access patient and scheduling information using industry standard technologies that provide real-time bi-directional communication with the EHR system (Layer 3), and patient data stored therein.

The integration with an EHR system may allow the middleware layer to extract information stored in patient records, update patient records and extract available appointment slots. All components of the AI²VR system (Layers 1, 2 and 3) may operate (e.g., respond to caller input, access and present information extracted from an EHR system) within time and performance limits that are commercially reasonable for a system whose goal is to mimic live human-to-human interaction.

The AI²VR system may provide an administrative interface. The administrative interface may provide tools for configuring custom slot filtering rules that define operating and scheduling optimization criteria. Illustrative slot filtering rules may control appointment slots presented to a caller based on: providers that work at a target office location, a provider's scheduling preferences, a patient's age or other restrictions on which type of patients can be examined by a provider, insurance plans accepted by a provider, or services available at the dialed office.

The AI²VR system may monitor caller interaction with the system. Illustrative caller interactions may include caller service requests and appointment slot selections. The AI²VR system may monitor system generated responses. Illustrative system generated responses may include appointment slots or other choices presented to a caller. The AI²VR system may include a dashboard for visualizing tracked statistics and reports based on analysis of logged/tracked caller behavior and system performance metrics.

The AI²VR system may monitor caller-system interactions and log corresponding performance metrics. The monitoring and logging may be valuable when evaluating the effectiveness of the AI²VR system and scheduling optimization algorithms. Monitoring performance metrics may enable healthcare providers to improve delivery of their healthcare services, optimize their scheduling systems, and enhance patient satisfaction.

The AI²VR system may log/track caller behavior and performance metrics for each layer. For example, with respect to the telephony layer (Layer 1), the AI²VR system may track and log duration of each call, a transcript of each call, an audio recording of each call, how many times has the caller dialed into the system, resolution of the phone call, abandonment rate (when and how often do callers leave the AI²VR system before completing a task), transmission latency when communicating with the EHR and middleware layers.

In parallel with a voice call, the AI²VR system may send text messages or pictures of information being discussed on call. For example, the telephony layer may send a caller a message (text or picture) showing various appointment choices that are available for selection. The text message may be color coded to make it easier to see the different appointment slot choices. For example, each appointment slot choice may be presented in a different color and font.

The transmitted text message may include touch sensitive hyperlinks that can be touched by the caller to select an appointment slot choice. The AI²VR system may detect that the caller has selected an appointment slot choice by touching the hyperlink and instruct the telephony or middleware layer to move to the next step in a scheduling workflow.

In parallel with voice call, the AI²VR system may initiate a video conference with a caller. For example, if the AI²VR system detects that a caller seems to be having trouble locating a desirable appointment, the AI²VR system may initiate a video call with a human agent to help the caller schedule an appointment. In some embodiments, the AI²VR system may initiate a video call with an AI model that is configured to visually show the caller appointment slot choices.

The AI²VR system may be configured to send automated voice reminders about upcoming appointments. The AI²VR system may allow patients to interact with the telephony layer to confirm, reschedule or cancel an appointment. During a reminder call, the patient may be provided access to office information or any other options and menus.

The AI²VR system may include an AI model configured to dynamically adjust voice properties used to interact with a caller. The AI model may dynamically determine which voice or language should be used to interact with the caller. The AI model may dynamically change an initially selected voice or language during the conversation based on detected caller voice inputs. Illustrative voice properties that may be dynamically adjusted by the AI model may include male voice, female voice, accent, tenor, cadence, tone, pitch, language, volume, spunk, humor, and vocabulary.

For example, if the AI model detects that a caller is getting upset, it may slow down the voice output responses or select voice properties configured to deescalate the tension. The AI model may determine that a caller is having trouble hearing voice outputs generated by the AI²VR system. The AI²VR system may adjust the loudness, tone, pitch or other audio attributes to improve the likelihood of the caller hearing the voice outputs.

During a video call, the AI model may dynamically adjust voice output properties based on how the caller looks, caller motions or how caller speaks on video. Based on content of caller voice input, the AI model may adjust voice output properties. For example, if a caller says something unfortunate occurred, the AI model may employ a sympathetic sounding voice or present sympathetic message.

The middleware layer may communicate with the EHR system using one or more APIs. If API access to an EHR system is not available, the appointment booking system may still provide caller with available appointment slots by extracting data (e.g., scraping) from screenshots of schedule displayed in EHR system to identify open appointments slots. This will allow patients to schedule appointments using voice commands regardless of functionality provided by the EHR system.

The AI²VR system may accurately confirm caller information such as name, phone number, birthdate and insurance. Accurate capture and confirmation of such information may reduce a number of duplicate patient records created when scheduling appointments over the phone or even when scheduling online. Unintentional duplicate records may be created when a patient or office staff member enters patient information incorrectly. Duplicate records are hard to both find and merge.

Using the appointment booking system, a caller may be presented with a proposed spelling of their name and asked to confirm the spelling. The confirmation may be presented to the caller via voice, text message or video chat. An AI model may apply fuzzy logic to identify and eliminate potential duplicate patient records. The AI²VR system may generate office forms and pre-populate those forms with confirmed spellings of a patient's name, insurance and other information that has been provided by a caller during a phone call.

The AI²VR system may verbally explain to a caller why a presented appointment slot is advantageous. For example, the system may explain that there is expected to be nice weather on the day or time of the available appointment. Or, the system may explain that traffic is expected to be light based on the presented appointment day/time and expected travel path the caller will take from their home or work address to the dialed office. Or, the system may explain that a wait time is expected to be short for the presented appointment.

A caller may be prompted to provide a voice input explaining why they are booking, rescheduling or cancelling an appointment. The captured voice input may be processed to determine how to classify the appointment or determine a duration of the appointment. For example, based on the provided reason, the AI model may determine whether a caller has asked to schedule a medical or cosmetic appointment. The AI model may identify a provider that has the expertise to service the medical needs of the caller.

The AI model may generate various personas and create competition among those personas to achieve the highest number of appointment bookings within a defined timeframe. The persona interacting with a caller may ask the caller to complete a survey and explain that completing the survey will help the persona win the competition against the other personas.

The AI²VR system may allow a provider or other office staff member to record voice input describing their schedule. The AI model may generate a schedule based on the recorded voice input received from the provider describing the schedule. The generated schedule may then be synced and integrated with the EHR system. For example, a provider may speak to the AI²VR system “I will see patients on Monday, Wednesday and Friday from 10 am-1 pm. The AI²VR system may pass the captured voice input to the AI model. The AI model may generate the verbally described schedule so that patients are thereafter only booked for the given provider during the described days and times.

If a provider needs to cancel appointments, a message can be sent to the AI²VR system. The AI²VR system may automatically trigger phone calls to those patients who need to be rescheduled or informed of the delay/cancellation.

Based on which provider is associated with the appointment selected by a caller, the caller may be played a personal audio or video message from the provider they are expected to visit. The provider message may give the caller a personal greeting at conclusion of an appointment booking call.

The AI²VR system may prompt a caller to complete a satisfaction survey after concluding a caller interaction. Prompting callers to complete a satisfaction survey after concluding a caller interaction is an important step in evaluating patient satisfaction using the AI²VR system and identifying areas for system improvement. This feature can help healthcare providers refine their scheduling systems and enhance the overall patient experience.

A technical challenge to AI model reliability is its tendency to “hallucinate” and produce unreliable responses to inputs. The AI²VR system may solve this technical problem by using a hybrid solution that leverages use of an AI model to confirm selection of an appropriate structured voice response. The AI model may be asked to check structured voice response selected by the AI²VR system using non-AI methods. Illustrative non-AI methods may include identifying keywords in a voice input or attempting to match received voice inputs to a predefined set of choices. Such a hybrid system may provide reliable and natural sounding healthcare applications without risk of hallucinations.

The overall goal of the AI²VR system is to provide natural, human sounding voices (e.g., voice speed, female, male, with local accents) and interact with callers using multiple languages, over multiple communication channels (voice, text, video) to schedule appointments, request office information and complete other self-service tasks. Interaction with a caller may begin by the receiving voice or text input to the AI²VR system and prompting the caller to choose a self-service task (e.g., appointment booking) they want to accomplish.

The AI²VR system may utilize voice input provided by the caller to provide personalized responses (e.g., use caller's first name or show understanding of the task the caller wants to complete) and overall mimic a personal human-to-human interaction. The telephony layer (Layer 1) may prompt a caller to provide any necessary voice input to execute a desired appointment task. For example, if the caller wants to book an appointment, the telephony layer may prompt for caller's name, address, birthdate, desired provider, desired office location, insurance information and when the caller wants an appointment.

The telephony layer may pass information captured from a caller to the middleware layer (Layer 2). The middleware layer may use the information captured by the telephony layer (Layer 1) to interact with the EHR system (Layer 3). The middleware layer may apply filtering rules that are specific to the medical practice, desired provider or office the caller has dialed.

The AI²VR system may log any suitable data elements for later analysis. Illustrative data elements may include: time each call is received, calling and dialed number, whether a call was received from a landline or mobile number, number of abandoned calls, at what point in a workflow was the call abandoned (e.g., in response to which question or prompt), duration of each call, time delay between voice prompts and receiving caller voice inputs, voice type/language/gender used during the call (e.g., male voice speaking Spanish), number of appointments booked, changed or cancelled over a defined time period (e.g., per hour, day, week, provider, office location, etc.)

In addition to using natural language, the AI²VR system may utilize multiple communication channels to interact with a caller. The AI²VR system may dynamically activate a communication channel based on context of a conversation and how a caller would most efficiently process information. For example, appointment slot choices may be more efficiently presented to callers visually rather than audibly. Therefore, while a phone call is in progress, the AI²VR system may text the caller available appointment choices and then have the caller respond (audibly, by touching a presented choice or by sending a responsive text message) with the choice they want to select. The AI²VR system may also ask a caller whether they prefer to hear choices audibly or visually. For example, a caller may be driving or have a visual disability and prefer to hear the appointment choices or other options audibly.

After booking an appointment, the AI²VR system may text the caller a summary about the appointment. The summary may include details about the time and date of the appointment and provider. After a caller books an appointment, the AI²VR system may text the caller links to office forms that need to be filled out beforehand. The text message may include a link for the caller to upload an image of their insurance card or driver's license. Information extracted information from the image of the insurance card or driver's license may be used by the middleware layer to formulate EHR system (Layer 3) requests.

The middleware layer may extract data from the insurance and submit that extracted information to a clearinghouse to verify the caller's insurance coverage. The middleware layer may abort and automated appointment scheduling workflow if the caller's coverage has lapsed. Aborting calls may be transferred to a human agent.

The middleware layer (Layer 2) may define operating and optimization criteria for the appointment booking system based on specific business rules. This feature allows for tailored solutions that cater to the unique needs of each medical practice or organization. The appointment booking system aims to facilitate rapid appointment booking in under 5 minutes (preferably under 3 minutes). To achieve this, the AI²VR system may incorporate custom filtering rules specific to a medical practice or organization.

The filtering rules may impact various stages of the appointment booking process. The middleware layer may be coupled with an AI model to In addition to the fuzzy logic component to reduce duplicate patient records referenced earlier, factors include: business intelligence and rules for reducing no shows and optimizing scheduling, matching office schedule optimization with factors desirable by the caller, providers available at an office location, time of day, and local factors such as weather and current traffic conditions and overbooking allowances based real-time or historical data.

The middleware layer may be coupled to an AI model. The AI model may dynamically respond to fluctuations in the real-time availability of open appointment slots for an office location at a given time of day. The AI model may also account of factors local to a caller, such as weather and traffic conditions and the caller's ability to reach the office during their desired appointment time.

The middleware layer may assess the caller's insurance coverage to confirm provider coverage and suggest providers who accept the same plan to reduce potential financial burdens. The middleware layer may apply filtering rules to provide callers with appointments choices with providers that are available at a dialed office location, at the caller's desired appointment date/time and accept the caller's insurance. The middleware layer may also apply filtering rules that account for provider age or other restrictions when attempting to match a caller with available appointment slots.

Filtering rules may be defined by office-specific requirements may include: provider specialties, ramp or elevator accessibility, and insurance accepted by providers at the dialed office location to optimize scheduling and reduce no-shows. A flexibility of the middleware layer to leverage decision making of an AI model and filtering rules set based on office-specific requirements maximizes the caller experience and practice efficiency.

The middleware layer may determine which office location to associate with each caller. The middleware layer may determine an office location based on a phone number dialed by the caller. The middleware layer may instruct the AI²VR system to prompt the caller to choose a specific location (e.g., if caller dialed general office number, corporate office or 800 number). An AI model associated with the middleware layer may assign an office location to the caller based on office locations previously visited by caller.

If a desired appointment is not available, the middleware layer may apply a filtering rule to offer alternatives based on patient wishes and office needs. The AI²VR system may provide voice output to the caller explaining that the desired appointment is not available and offer 1-3 alternative appointment options. The middleware layer may select the alternative appointment options. If very few appointments are available at a target office location, a filtering rule may switch to a different office location within a predefined radius of the dialed office location.

The filtering rules may present appointment choices based on a VIP status of the caller. The status may be determined based on examining information stored in the patient's record obtained from the EHR system. For example, the AI²VR system may allow overbooking for a caller with VIP status. The filtering rules may allow overbooking for a caller with a specific medical condition. For example, the AI²VR system may make certain otherwise restricted times or providers available to a caller that has a critical or life-threatening condition. The AI²VR system may allow certain services or products to be available to callers that have VIP status, a medical condition or other attribute stored in a patient record.

The middleware layer may classify caller as a “new” patient (no patient record in EHR) or as an “established” patient (successfully locate patient record in EHR). A filtering rule applied by the middleware layer may determine how often to ask callers to update their insurance information. The middleware layer may apply filtering rules based on an appointment type. Illustrative appointment types may include: medical, non-medical (suture removal, follow up wound care), surgery, cosmetic and telehealth. The telephony layer may prompt a caller to provide an appointment type. In some embodiments, the AI model may determine an appointment type based on a natural language reason for scheduling an appointment provided by the caller.

The middleware layer may define a time range that will be searched for open appointments in the EHR system. The middleware layer may define one or more business rules that define a scheduling algorithm. An illustrative business rule may assign different weights to appointment selection parameters. Illustrative parameters may include a target calendar date, day of week, time of day, provider and/or location.

An appointment matching all requested parameters provided by a caller may not be available. The middleware layer may automatically determine relevant alternatives based on parameters captured from the caller. For example, if there are no afternoon appointment slots with Dr. Z on Thursday, the middleware layer may present alternative slots that include or more of the parameters provided by the caller such as an appointment with Dr. X on Thursday afternoon or with Dr. Z on Wednesday morning.

The middleware layer may determine which appointment slots are presented to a caller based on a caller's insurance plan information. The middleware layer may only search the EHR system for appointments with providers that participate in the caller's insurance plan. In some embodiments, the caller may not be prompted to specify a desired time for an appointment. The middleware layer may search automatically for available appointments based on parameters associated with the caller's last three appointments. Based on the last three appointments, the middleware layer may automatically determine a template that includes parameters for booking a new appointment on behalf of the caller.

The middleware layer may determine whether to present target information to caller about consequences of cancelling an appointment (e.g., whether cancelling the appointment will incur a fee) and instruct telephony layer (Layer 1) to inform caller of those consequences. The middleware layer may determine whether a caller should be blocked or restricted from booking, rescheduling, or cancelling an appointment. A blocked caller may be transferred to a human agent to reschedule or cancel an appointment. A caller may be blocked from using the AI²VR system to reschedule or cancel an appointment within a threshold time (e.g., 24 hours) of the scheduled appointment time. In determining whether to impose a restriction, the middleware layer may take account of: number of reschedules or cancellations by the caller in the last month, the reason for the current appointment, current outstanding balance (outstanding balance will need to be pulled from EHR system) or time remaining until the scheduled appointment.

Via the AI²VR system, the middleware layer may receive an appointment selection from the caller and trigger syncing of any newly scheduled, changed, or cancelled appointments with the EHR system. In response to successfully booking an appointment, the AI²VR system may provide the caller access to forms that need to be completed by the caller before their appointment or other pre-appointment instructions. For example, after successfully booking an appointment, the AI²VR system may text the caller a link to the office forms. The AI²VR system may also send appointment reminders to the caller at a predetermined time before an upcoming appointment.

The AI²VR system may determine whether an office billing department or call center is open before transferring a caller to that department. The AI²VR system may schedule callbacks for a specific administrative department.

The middleware layer may coordinate bi-directional communication with the EHR systems (Layer 3). Such bi-directional communication may include: pulling open appointments slots, confirming new appointments, pushing newly booked, rescheduled or cancelled appointments, pulling patient medical records, updating patient medical implement records, appointment waitlist functionality by periodically checking an EHR schedule, locating appropriate open appointment slots for a caller, ability to accept payments over the phone or via text, automated verification of insurance information, providing an administrator interface to customize filtering rules.

The AI²VR system may include an intuitive, easy to use administrative user interface for configuring custom filtering rules. Illustrative filtering rules that may be set for each office location include: default appointment scheduling rules (e.g., break times, when to schedule new patients), weekend, holiday, walk-in scheduling rules, appointment categories and service provided, whether or not a fee is applied for cancellation within 24-hours and amount of the fee, specific diagnosis(es) or procedures treated at target location, provider attributes (specialties, participating insurance, available locations, overbooking rules, hours and schedule, patient type restrictions).

The AI²VR system may integrate with an EHR system. The integration with an EHR system provides real-time bi-directional communication that: extracts patient information from records stored in the EHR system, pushes updates to patient records and schedule information, extracts open appointments slots over a defined time interval from the EHR system, and updates the schedule maintained by the EHR system.

The integrations with EHR systems provide may provide the AI²VR system the ability, in real-time to: pull open appointments from an EHR system, search for a target appointment slot requested by a caller, locate a patient record and book an appointment in response to a caller selection, push newly booked, rescheduled or cancelled appointments to the EHR system, based on information obtained from a caller (e.g., name, phone number, birthdate) search for and obtain a corresponding patient record.

When the EHR system contains a patient record for a caller, the middleware layer may obtain the following information from the EHR system: patient ID (as used by EHR system), patient Name (First, Last), gender, birthdate, upcoming open appointment(s) (if any), insurance information, attributes of caller's three most recent appointments (provider, location, day of week, time and duration), payment information (payment on file and current balance), most recent diagnosis, special patient needs (disability or language requirements), other information as may be available from the EHR system.

Illustrative embodiments of apparatus and methods in accordance with the principles of the invention will now be described with reference to the accompanying drawings, which form a part hereof. It is to be understood that other embodiments may be utilized, and structural, functional and procedural modifications may be made without departing from the scope and spirit of the present invention.

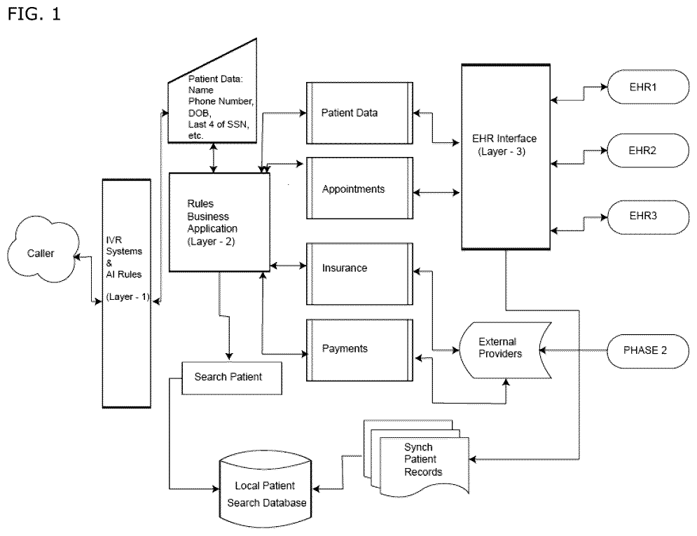

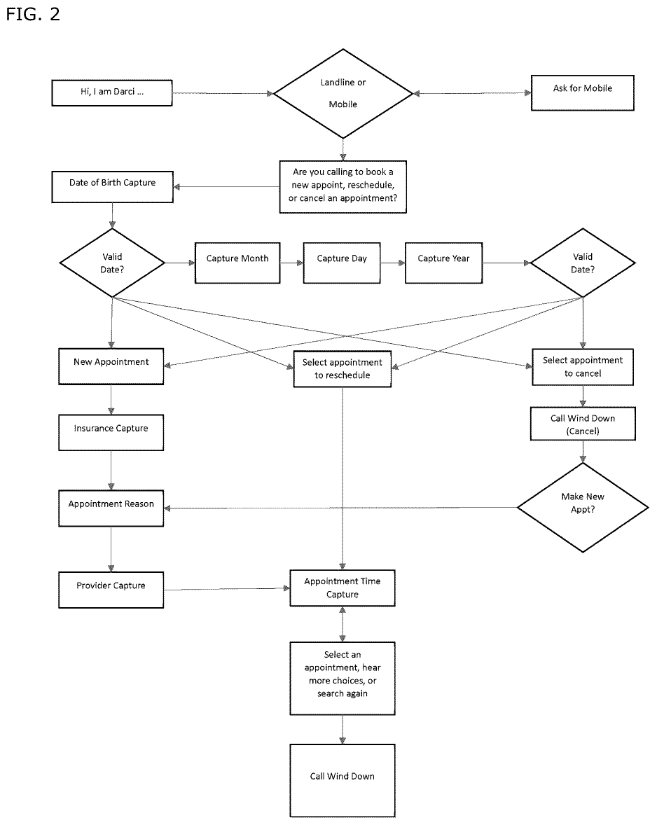

FIG. 1 shows an illustrative system architecture in accordance with the principles of the description. FIG. 2 shows an illustrative process that may be implemented by an appointment booking system in accordance with the principles of the description.

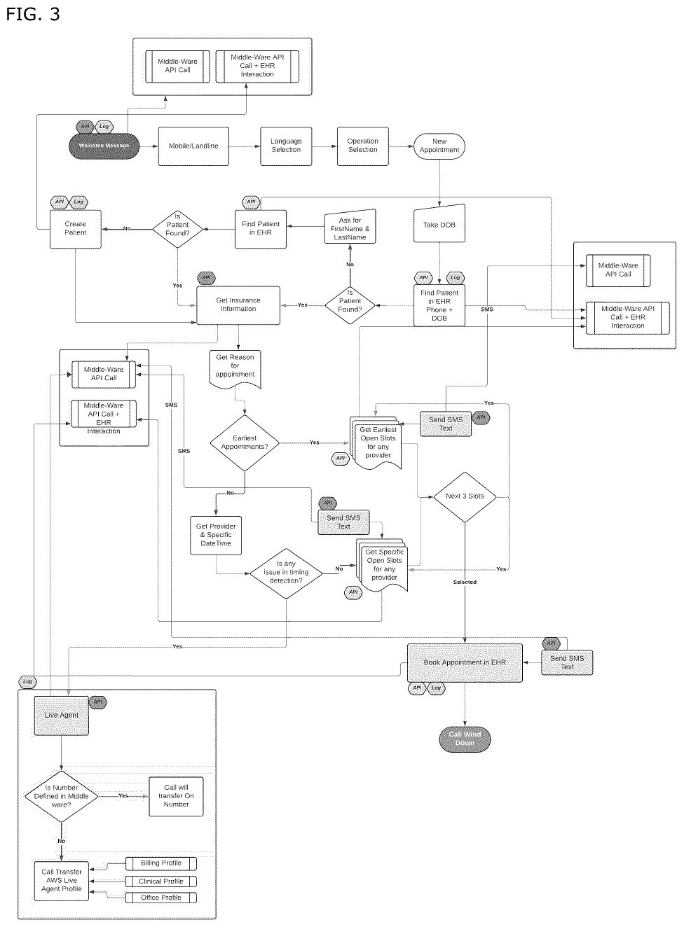

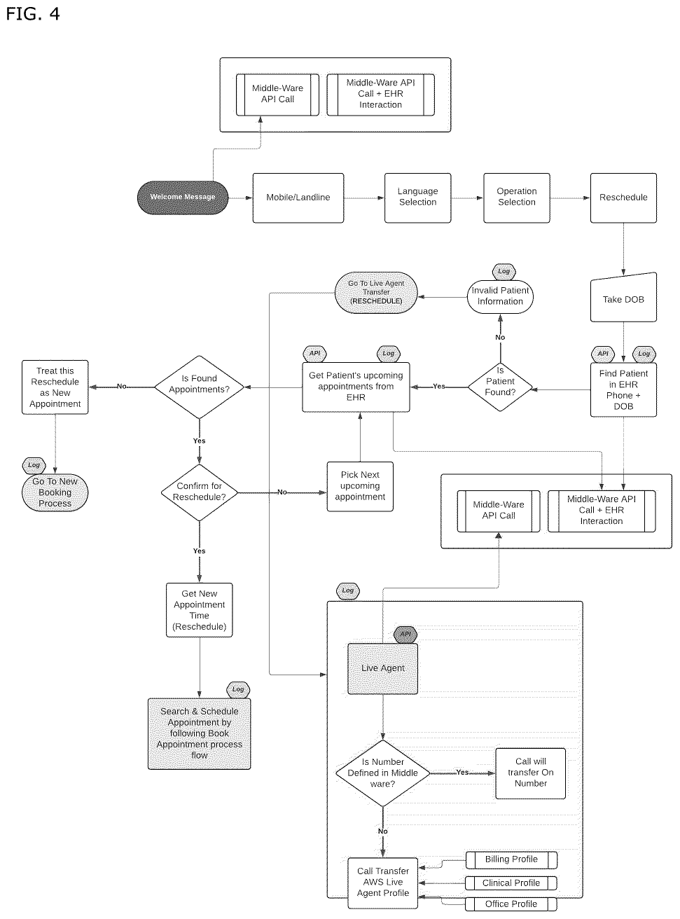

FIG. 3 shows an illustrative process for booking a new appointment, components and functions that that may be implemented by an appointment booking system in accordance with the principles of the description. FIG. 4 shows an illustrative process for rescheduling an appointment, components and functions that that may be implemented by an appointment booking system in accordance with the principles of the description. FIG. 5 shows an illustrative process for cancelling an appointment, components and functions that that may be implemented by an appointment booking system in accordance with the principles of the description.

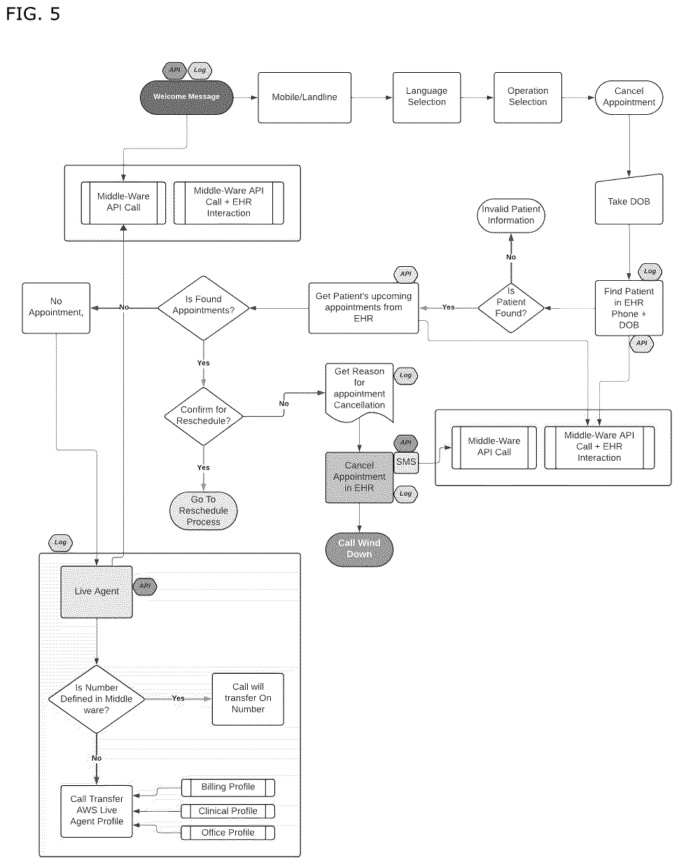

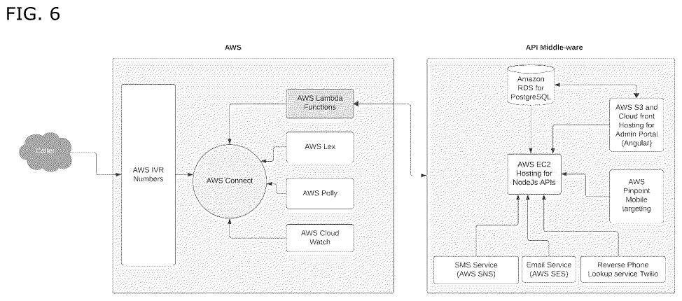

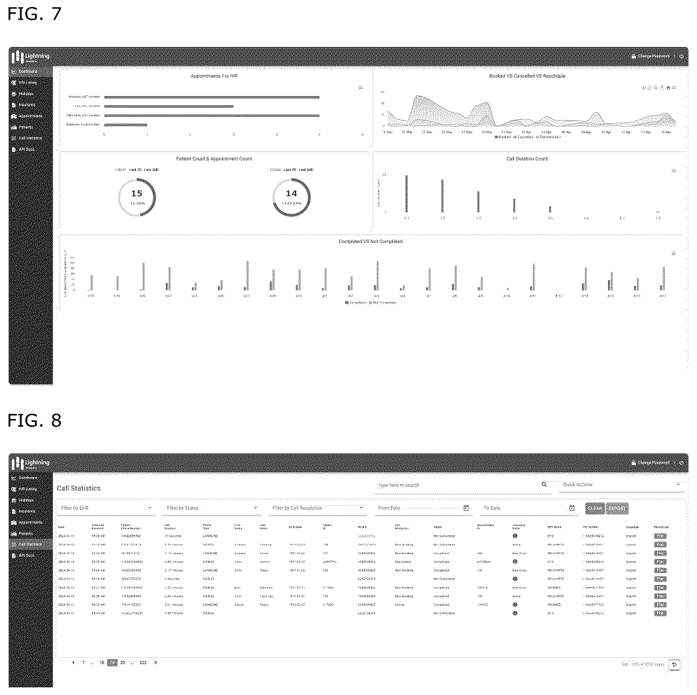

FIG. 6 shows an illustrative system architecture for an appointment booking system in accordance with the principles of the description. FIG. 7 shows an illustrative graphical user interface for viewing activity tracked by an appointment booking system in accordance with the principles of the description. FIG. 8 shows another illustrative graphical user interface for viewing activity tracked by an appointment booking system in accordance with the principles of the description.

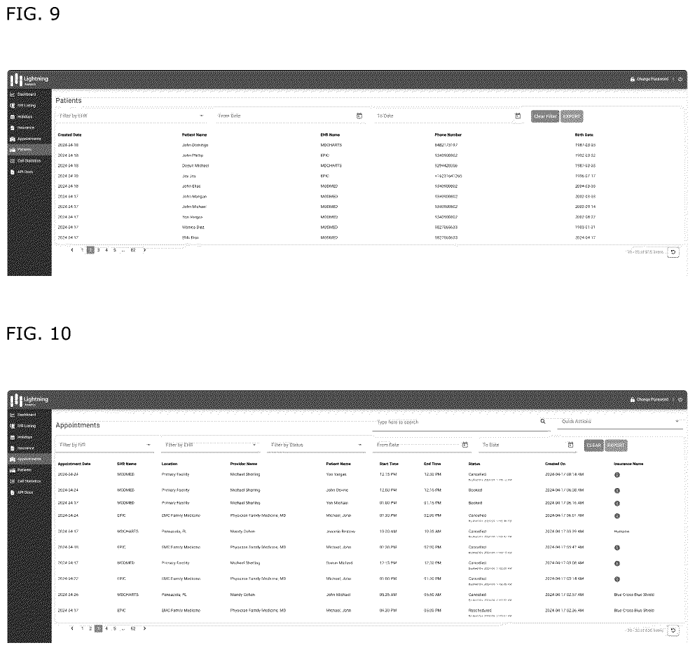

FIG. 9 shows an illustrative graphical user interface for viewing new patients that have been registered by an appointment booking system in accordance with the principles of the description. FIG. 10 shows an illustrative graphical user interface for viewing appointment activity processed by an appointment booking system in accordance with the principles of the description.

Thus, apparatus and methods for ARTIFICIAL INTELLIGENCE APPOINTMENT SCHEDULING SYSTEM are provided. Persons skilled in the art will appreciate that the present disclosure can be practiced in embodiments other than the described embodiments, which are presented for purposes of illustration rather than of limitation.

What is claimed is:

1. An AI system for scheduling appointments, the AI system comprising:
a telephony layer that is configured to:
receive a voice input from a caller; and
compute an intent of the caller based on the received voice input; and
a middleware layer that is configured to formulate a set of available slots for the caller based on the intent.

2. The AI system of claim 1, wherein the middleware layer is configured to utilize a scheduling algorithm to formulate the set of available slots.

3. The AI system of claim 2, wherein the scheduling algorithm is customizable for a target medical office.

4. The AI system of claim 1 wherein the set of available slots is formulated by a machine learning algorithm.

5. The AI system of claim 4, wherein the machine learning algorithm is trained using historical scheduling data associated with the target medical office.

6. The AI system of claim 1 wherein the telephony layer is configured to receive the voice input from the caller as a natural language statement.

7. The AI system of claim 1 wherein:
the intent of the caller includes at least one appointment selection parameter; and
the middleware layer is configured to select the set of available slots such that each member of the set matches at least one appointment selection parameter.

8. The AI system of claim 1 wherein:
the intent of the caller includes at least one appointment selection parameter; and
the middleware layer is configured to select the set of available slots such that each member of the set is linked to at least one appointment selection parameter.

9. The AI system of claim 1 wherein:
the intent of the caller includes an appointment selection parameter; and
the middleware layer is configured to select the set of available slots such that each member of the set is associated with the appointment selection parameter.

10. The AI system of claim 1 wherein:
the intent of the caller includes a plurality of appointment selection parameters; and
the middleware layer is configured to select the set of available slots such that each member of the set is associated with at least one of the plurality of appointment selection parameters.

11. The AI system of claim 10, wherein the middleware layer is configurable such that:
different weights are assignable to each of the plurality of appointment selection parameters; and
the set of appointment slots is determined based on filtering available slots using the different weights.

12. The AI system of claim 10, further comprising a web portal that provides a user interface for assigning the different weights to each of the plurality of appointment selection parameters.

13. A computer program comprising instructions stored on a tangible medium that when executed on a processor:
answers a telephone call received from a caller;
prompts the caller using natural language to request a service; and
in response to detecting that the caller requested an appointment scheduling service, communicates with an electronic health record (“EHR”) system to provide the requested appointment scheduling service to the caller during the telephone call.

14. The computer program of claim 13 further comprising instructions, that when executed by the processor:
filter a set of appointment slots; and
generate a subset of the appointment slots.

15. The computer program of claim 14 further comprising instructions, that when executed by the processor filter the set of appointment slots based on a set of configurable appointment selection parameters.

16. The computer program of claim 15 further comprising instructions, that when executed by the processor dynamically determine the set of configurable appointment selection parameters.

17. The computer program of claim 15 further comprising instructions, that when executed by the processor dynamically determine the set of configurable appointment selection parameters based on the caller's previously scheduled appointments.

18. The computer program of claim 15 further comprising instructions, that when executed by the processor dynamically determine the set of configurable appointment selection parameters using a machine learning algorithm.

19. An automated system for scheduling an appointment during a phone call, the automated system comprising:
an artificial intelligence interactive voice response (“AI²VR”) system configured to receive a first voice input from a caller; and
a middleware layer configured to:
interact with an electronic health record system and obtain a set of available appointment slots;
using a set of filtering rules, formulate a subset of the available appointment slots;
trigger the AI²VR system to present the subset to the caller; and
schedule a target appointment slot for the caller based on a second voice input received from the caller by the AI²VR system.

20. The automated system of claim 19 further comprising an administrative portal that provides a user interface for configuring the set of filtering rules.
